(19) United States
(12) Patent Application Publication
(10) Pub. No.: US 2009/0191617 A1
(43) Pub. Date: Jul. 30, 2009

(54) BIO CHIP AND RELATED TECHNOLOGIES INCLUDING APPARATUS FOR ANALYZING BIOLOGICAL MATERIAL

(75) Inventors: Gueisam LIM, Seoul (KR); Gyoung Soo KIM, Seoul (KR); Seok Jung HYUN, Seoul (KR); Yeonjae KANG, Seoul (KR); Yunhee KU, Seoul (KR); Seong Moon CHO, Seoul (KR); Hyungki HONG, Seoul (KR)

Correspondence Address: FISH & RICHARDSON P.C., P.O. BOX 1022, MINNEAPOLIS, MN, 55440-1022, US

(73) Assignee: LG ELECTRONICS INC., Seoul (KR)

(21) Appl. No.: 12/046,536

(22) Filed: Mar. 12, 2008

(30) Foreign Application Priority Data
Jan. 25, 2008 (KR) 10-2008-0007817

(51) Int. Cl. C12M 1/34

(52) U.S. Cl. 435/288.7; 435/287.1

(57) ABSTRACT

The bio chip and the apparatus for analyzing biological material are disclosed, the bio chip and the apparatus being capable of analyzing a variety of specific materials included in biological materials using a single bio chip injected with a single biological material, capable of conducting an optical measurement and an electro-chemical measurement to the enhancement of efficiency, capable of forming a sterilizer at the bio chip to enable a swift disinfection of vulnus caused by blood collection to the convenience of a user, and capable of mounting a laser beam source at the apparatus to enable a swift blood collection, wherein the apparatus is provided with a transfer unit for transferring the bio chip having a sterilizer, whereby the bio chip is transferred following the blood collection to enable automatic disinfection and analysis.

### CROSS-REFERENCE TO RELATED APPLICATION

The present application is based on, and claims priority from, Korean Application Number 10-2008-0007817, filed Jan. 25, 2008, the disclosure of which is incorporated by reference herein in its entirety.

### FIELD

The following description relates generally to a bio chip and an apparatus for analyzing biological material capable of analyzing a variety of specific materials included in biological materials using a single bio chip, and capable of automatically conducting blood-collecting, sterilization and analysis.

### BACKGROUND

A bio sensor may include a series of devices for immobilizing molecules having a biological activity on the surface of a solid small thin film by utilizing covalent bonding or non-covalent bonding and for changing interactions or bonding in biological materials to an electrical signal useful for monitoring and assaying gene expression, gene mutation, gene polymorphism and the like.

Bio sensors may also be called bio chips in a broader sense that includes micro devices for assaying biological molecules quantitatively and qualitatively. Bio chips may be categorized into three types based on thin film material formed on a solid substrate and targets to be assayed, that is, a DNA chip, a cell chip and a protein chip.

### SUMMARY

Structures for assaying a single specific material included in biological materials are described, which may compare to conventional bio chips by increasing and/or otherwise improving production efficiency, yield, and applicability and while reducing wasting of raw materials.

In one general aspect, a bio chip for analyzing biological material comprises: a substrate; a protection film formed on the substrate with first through holes for exposing the substrate, micro channels each connected to the first through holes, and an inlet port into which biological materials are injected by being connected to the micro channels; and reaction-inducing materials each immobilized on the substrate exposed to the first through holes.

In another general aspect, a bio chip for analyzing biological material comprises: a substrate formed with electrode pads and electrode lines each connected to the electrode pads; a protection film formed on the substrate exposing the electrode pads and mounted with through holes exposing distal ends of the electrode lines, micro channels each connecting the through holes, and an inlet port connected to the micro channels and into which biological materials are injected; and reaction-inducing materials immobilized on the distal ends of the electrode lines exposed to each through hole.

In another general aspect, an apparatus for analyzing biological material comprises: a connector formed with reaction regions in which specific materials and reaction-inducing materials included in the biological material are reacted, formed with distal ends of electrode lines on part of the reaction regions and connected to electrode pads of bio chip having electrode pads connected to the electrode lines; an electro-chemical measurer applying a voltage to the electrode pads of the bio chip via the connector to measure a current variation value in response to the applied voltage, converting the current variation value to an electrical signal and outputting the electrical signal; a photo sensor irradiating light on reaction regions where the distal ends of the electrode lines of the bio chip are not formed, and collecting the light reflected or transmitted therefrom; an optical measurer measuring a light intensity collected from the photo sensor, converting the light intensity to an electrical signal and outputting the electrical signal; and an analyzer receiving the signal outputted from the electro-chemical measurer and the optical measurer to qualitatively and quantitatively analyze the biological material.

In another general aspect, an apparatus for analyzing biological material comprises: a bio chip formed with reaction regions in which specific materials and reaction-inducing materials included in the biological material are reacted; and a biological material analyzer formed with the bio chip for measuring the reaction regions of the bio chip to qualitatively and quantitatively analyze the biological material, wherein the biological material analyzer comprises: a photo sensor irradiating light on the reaction regions and collecting the light transmitted or reflected therefrom; an optical measurer measuring a light intensity collected from the photo sensor, converting the light intensity to an electrical signal and outputting the electrical signal; and an analyzer receiving the signal outputted from the electro-chemical measurer and the optical measurer to qualitatively and quantitatively analyze the biological material.

Implementations of these aspects may include one or more of the following effects.

One single biological material can be injected into a single bio chip to analyze a variety of specific materials included in the biological materials.

Optical measurement and electro-chemical measurement can be simultaneously conducted to improve the efficiency.

The biological material can be supplied from an inlet port to a reaction region by way of a capillary phenomenon in micro channels, dispensing with any special manipulation from outside.

The bio chip can be formed with a sterilizer to allow any vulnus caused by, i.e., blood collection to be swiftly sterilized to the enhanced convenience to a user. Collected blood can be supplied to the reaction region upon blood collection to allow a swift analysis of the blood.

The bio chip is formed with a sterilizer for sterilizing any vulnus caused by blood collection and a treatment unit for treating the vulnus.

The apparatus for analyzing the biological material is formed with a laser beam source to enable a swift blood collection, and is also formed with a device for transferring the bio chip provided with the sterilizer to allow the bio chip to be transferred for automatic blood collection, sterilization and analysis.

### BRIEF DESCRIPTION OF THE DRAWINGS

FIG. 1 is a schematic plan illustrating a bio chip according to a first exemplary implementation.

FIG. 2 is a schematic plan illustrating another bio chip according to a first exemplary implementation.

FIGS. 3A and 3B are a schematic plan and a cross-sectional view illustrating still another bio chip according to a first exemplary implementation.

FIG. 4 is a cross-sectional view illustrating a bio chip formed on a top surface of a substrate according to the first exemplary implementation.

FIGS. 5A and 5B are a schematic plan illustrating micro channels formed on a protection film of the bio chip according to the first exemplary implementation.

FIG. 6 is a schematic plan illustrating a bio chip formed on a top surface of a substrate according to the first exemplary implementation.

FIG. 7 is a schematic plan illustrating an inlet port formed on a top surface of a bio chip according to the first exemplary implementation.

FIG. 8 is an exploded perspective view illustrating a detailed construction of a bio chip according to the first exemplary implementation.

FIG. 9 is a schematic perspective view illustrating a bio chip according to a second exemplary implementation.

FIG. 10 is a schematic plan illustrating another bio chip according to a second exemplary implementation.

FIGS. 11A and 11B are partial cross-sectional views illustrating a sterilizer of a bio chip according to a second exemplary implementation.

FIG. 12 is a partial cross-sectional view illustrating another sterilizer of a bio chip according to a second exemplary implementation.

FIG. 13 is a plan illustrating a bio chip according to a third exemplary implementation.

FIG. 14 is a schematic block diagram illustrating an apparatus for analyzing biological material of a bio chip.

FIG. 15 is a schematic partial cross-sectional view illustrating a state of an apparatus for analyzing biological material of a bio chip.

FIGS. 16A and 16B are schematic perspective views illustrating another state of an apparatus for analyzing biological material of a bio chip.

FIGS. 17A to 17D are schematic cross-sectional views illustrating a method for collecting blood from a bio chip of the second implementation immobilized on an apparatus for analyzing biological material.

FIGS. 18A to 18D are schematic cross-sectional views illustrating a method for collecting blood from a bio chip of the second implementation immobilized on an apparatus for analyzing another biological material.

FIG. 19 is a schematic concept representation illustrating an operation of transferring a bio chip from an apparatus for analyzing biological material.

### DETAILED DESCRIPTION

Hereinafter, a bio chip and an apparatus for analyzing biological material in accordance with the exemplary implementations will be described in detail referring to the accompanying drawings.

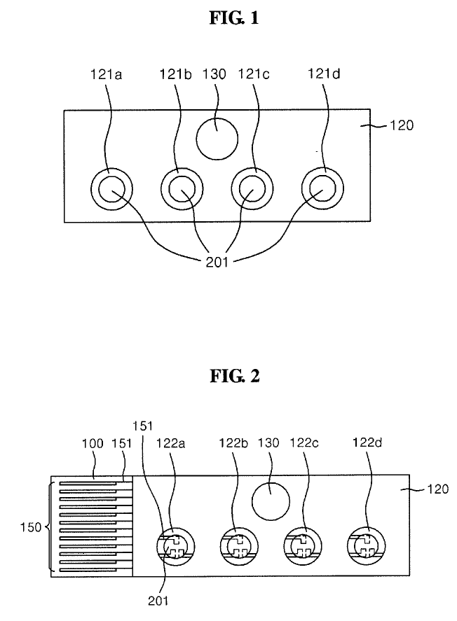

Referring to FIG. 1, a bio chip for analyzing biological material includes: a substrate; a protection film (120) formed on the substrate with first through holes (121a, 121b, 121c, 121d) for exposing the substrate, micro channels each connected to the first through holes (121a, 121b, 121c, 121d), and an inlet port (130) into which biological materials are injected by being connected to the micro channels; and reaction-inducing materials (201) each immobilized on the substrate at a position that is exposed to/through the first through holes.

It should be noted for reference that FIG. 1 does not illustrate the substrate and the micro channels.

In the bio chip, thus constructed, biological materials may be injected into the inlet port (130), and the biological materials may be supplied to the first through holes (121a, 121b, 121c, 121d) from the inlet port (130) via the micro channels. The biological materials supplied to the first through holes (121a, 121b, 121c, 121d) may be reacted with the reaction-inducing materials (201), where the reacted degree is optically measured, and the measured reacted degree is utilized to analyze the biological materials.

The reaction-inducing materials (201) may include different reaction-inducing materials. In other words, the reaction-inducing materials respectively located at a position on the substrate corresponding to and exposed to/through the first through holes (121a, 121b, 121c, 121d) may be respectively different reaction-inducing materials, which correspondingly react with various specific materials included in the supplied biological materials, whereby reactions may be optically measured from each of the first through holes (121a, 121b, 121c, 121d).

For example, if one reaction-inducing material reacts with cholesterol, and another reaction-inducing material reacts with hemoglobin, said one reaction-inducing material may react with the cholesterol contained in the biological material while said another reaction-inducing material may react with hemoglobin. Therefore, one biological material can be injected into one bio chip to effect analysis of various specific materials contained in the biological material. The biological materials may be, for instance, body fluid including blood, urine, serum and saliva.

Referring to FIG. 2, a bio chip for analyzing biological material comprises: a substrate (100) formed with electrode pads (150) and electrode lines (151) each connected to the electrode pads (150); a protection film (120) formed on the substrate (100) exposing the electrode pads (150) and mounted with second through holes (122a, 122b, 122c, 122d) exposing distal ends of the electrode lines (151), micro channels each connected the second through holes (122a, 122b, 122c, 122d), and an inlet port (130) connected to the micro channels and into which biological materials are injected; and reaction-inducing materials (201) immobilized on the distal ends of the electrode lines (151) exposed to each second through hole (122a, 122b, 122c, 122d). In other words, the bio chip works in such a fashion that the biological materials respectively supplied to the second through holes (122a, 122b, 122c, 122d) react with the reaction-inducing materials (201), and a reaction degree is electro-chemically measured.

The distal ends of the electrode lines (151) may be connected to the electrode pads (150), while the other ends of the electrode lines (151) may be dispersed to be positioned within the second through holes (122a, 122b, 122c, 122d).

A screen print may be employed to form pasted electrode material, which is plasticized at a predetermined temperature to form the electrode pads (150) and the electrode lines (151), or photolithography process may be used to form the electrode pads (150) and the electrode lines (151). The distal ends of the electrode lines (151) that are used for measurement may comprise varying sizes and shapes, and two or more electrodes may be used for each measurement case.

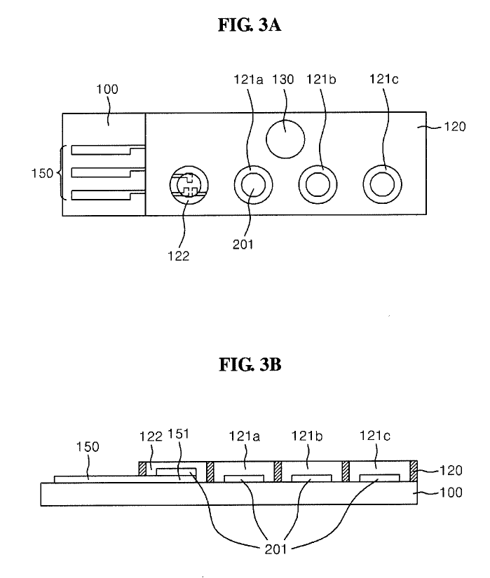

FIGS. 3A and 3B are a schematic plan and a cross-sectional view illustrating still another bio chip according to a first exemplary implementation, where the bio chip may be mixedly formed with the first through holes for optical measurement of FIG. 1, and formed with second through holes for electro-chemical measurement of FIG. 2.

In other words, a top of the substrate (100) of the bio chip as in FIG. 1 may be further formed with the electrode pads and electrode lines respectively connected to the electrode pads, the protection film may expose the electrode pads, and the protection film may be further formed with at least one or more second through holes exposing the distal ends of the electrode lines and micro channels connecting the second through holes and the inlet port.

As illustrated in FIG. 3A, the protection film (120) of the bio chip may be formed on the substrate (100) exposing the electrode pads (150), and is formed with the first through holes (121a, 121b, 121c) and second through hole (122).

As illustrated in FIG. 3B, the first through holes (121a, 121b, 121c) may contain only the reaction-inducing material (201), and the second through hole (122) contains the electrode lines (151) and the reaction-inducing material (201). Thus, the optical measurement and electro-chemical measurement can be simultaneously conducted to enhance the efficiency.

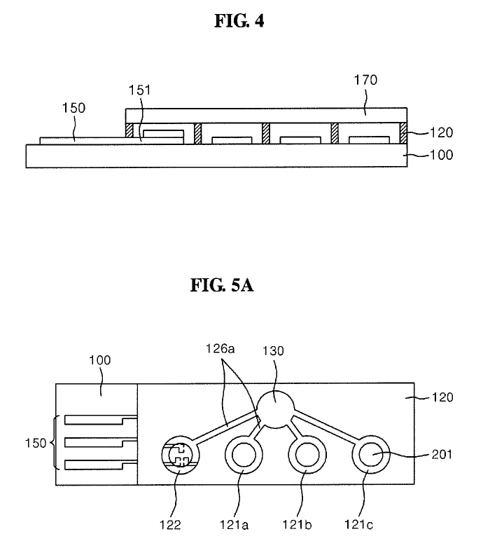

Referring to FIG. 4, the protection film (120) of the bio chip may be formed thereon with an upper substrate (170). The upper substrate (170) may be formed with a main inlet port communicating with the inlet port formed at the protection film (120) of the bio chip. The biological material may be injected into the main inlet port of the upper substrate (170), and the biological material injected into the main inlet port may pass through the inlet port formed at the protection film (120) and the micro channels to be supplied to the first and second through holes.

The substrate (100) formed underneath the bio chip and the upper substrate (170) may be transparent. In other words, one of the substrate (100) and the upper substrate (170) or both the substrates (100, 170) be made of transparent substrates to allow optically measuring the reaction of the biological materials.

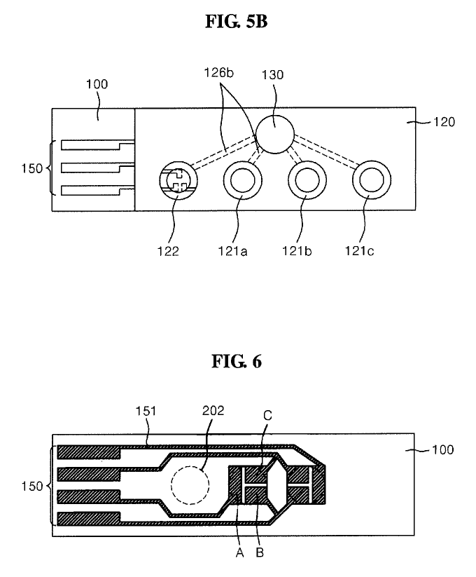

In FIGS. 5A and 5B, the micro channels of bio chip is formed inside or on the protection film. In other words, micro grooves may be formed on the protection film (120) to embody the micro channels (126a) as shown in FIG. 5A, and as depicted in FIG. 5B, micro paths may be formed inside the protection film (120) to embody the micro channels (126b). The width of each micro channel ranges from 0.1 mm˜1 mm, typically.

The length of each micro channels may be the same so that the biological materials can be uniformly supplied from the inlet port (130) to the first through holes (121a, 121b, 121c) and second through holes (122). The micro channels are manufactured using micro-fluidic control technique, such that biological materials can be supplied from the inlet port (130) to the first through holes (121a, 121b, 121c) and second through holes (122) according to capillary phenomenon without any special manipulation.

The micro-fluidic control technique separate blood corpuscles including red corpuscle and white corpuscle from blood elements including blood plasma but excluding cells, and the separated blood elements are supplied from the inlet port (130) to the first through holes (121a, 121b, 121c) and second through holes (122) via the micro channels. Notably, different of the capillaries may be differently configured to enable routing of different aspects of a single introduced biological material to different areas of the substrate for reaction with the same or different reaction-inducing materials located at those sites, yielding concurrently observable test results.

Referring to FIG. 6, the substrate (100) may be formed thereon with electrode pads (150) and electrode lines (151) each connected to the electrode pads (150).

Distal ends of the electrode lines (151) may be positioned at a substrate region formed with the second through holes for electro-chemically measuring the reaction degree of specific materials and the reaction-inducing materials included in the biological materials.

The distal ends of the electrode lines (151) may be configured in the form of pads (A, B, C) to easily detect the reaction degrees of the specific materials and the reaction-inducing materials contained in the biological materials.

The circular dotted lines (202) in FIG. 6 indicate a region where the reaction-inducing materials are immobilized. As shown, a substrate region where the single second through hole is formed is arranged with three electrode lines, where the three electrode lines are respectively a working electrode, a reference electrode and a counter electrode.

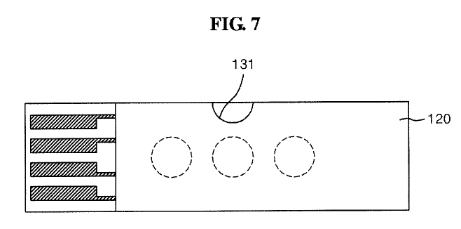

Referring to FIG. 7, an inlet port (131) of the bio chip may be formed at a lateral surface of the protection film (120). In the case where an upper substrate is formed on the protection film (120), a main inlet port may be formed at a lateral surface region of the upper substrate correspondingly opposite to the inlet port (131) formed at the protection film (120) to thereby enlarge an inlet port area.

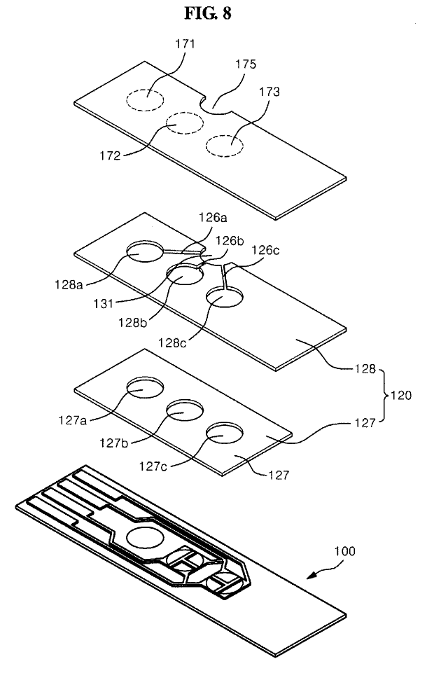

Now, referring to FIG. 8, the protection film (120) may include an isolation film (127) formed on the substrate (100) and a polymer film (128) formed on the isolation film (127). The isolation film (127) and the polymer film (128) may be formed with openings (127a, 127b, 127c, 128a, 128b, 128c) correspondingly opposite to the substrate (100) region for measuring the reaction degrees of the specific materials and reaction-inducing materials contained in the biological materials.

The polymer film (128) may be laterally formed with an inlet port (131), and the polymer film (128) may be formed with micro channels (126a, 126b, 126c) for connecting the inlet port (131) to the openings (128a, 128b, 128c) formed at the polymer film (128).

In a case where the protection film (120) is formed thereon with an upper substrate (170), a main inlet port (175) may be formed at a lateral surface of the upper substrate (170) correspondingly opposite to the inlet port (131) formed at the protection film (120).

As shown, the upper substrate (170) is formed with a transparent substrate through which light can pass. Alternatively, an opaque material film may be formed at the upper substrate (170), such that light can pass through only the region correspondingly opposite to the openings (127a, 127b, 127c, 128a, 128b, 128c) formed at the isolation film (127) and the polymer film (128).

As a result, the upper substrate (170) may be formed with light permissible regions (171, 172, 173) for passing irradiated light through for measuring the reaction degrees of the specific materials and reaction-inducing materials contained in the biological materials. The isolation film (127) may be formed at regions except for the distal ends of the electrode lines and the electrode pads for measurement.

The polymer film (128) may be embodied by forming at the isolation film (127) with a double-sided tape coated with adhesive polymer materials, or by printing on the isolation film (127) with polymer film materials.

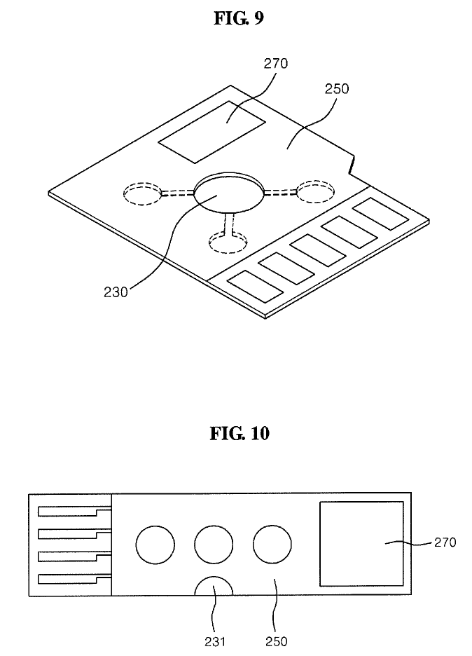

FIG. 9 is a schematic perspective view illustrating a bio chip according to a second exemplary implementation, where the bio chip according to the second implementation further includes a sterilizer (270) in addition to the chip structure of the first implementation.

To be more specific, the sterilizer (270) is formed on an upper substrate (250) in the bio chip structure formed with the upper substrate on the protection film, whereby blood is collected by a user who injects the collected blood into an inlet port (230) of the bio chip, and the finger vulnus caused by the blood collection can be sterilized by the sterilizer (270) provided at the bio chip.

With the sterilizer formed at a bio chip, blood may be injected the moment the blood is collected to assay the blood, and a finger vulnus caused by the blood collection may be swiftly sterilized by a sterilizer, allowing implementing a variety of functions with a single bio chip to the convenience of a user.

For reference, the inlet port (230) is formed in the shape of a through hole that has penetrated the upper substrate (250) and the protection film. Furthermore, FIG. 10 is a schematic plan illustrating a state of the sterilizer (270) provided at a bio chip, where the inlet port (231) into which the biological materials are injected is formed at a lateral surface of the upper substrate (250) and the protection film.

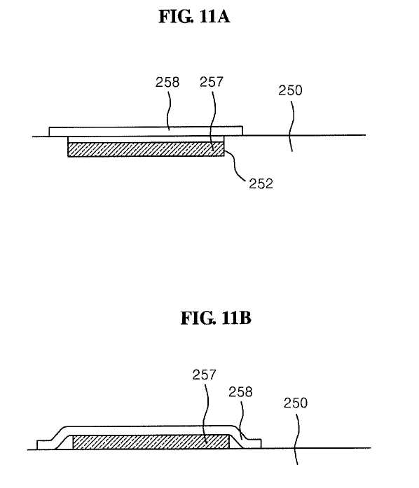

Now, referring to FIGS. 11A and 11B, the sterilizer of the bio chip may include a groove (252) formed on the upper substrate (250), and a sterilization material (257) filled inside the groove (252) (see FIG. 11A).

The sterilizer may also include a cover layer (258) covering the groove (252) and adhered to the upper substrate (250).

If the cover layer (258) is further included at the sterilizer, leakage of sterilization material (257) from the sterilizer can be prevented, and if a user is to conduct sterilization, the sterilization can be performed at the sterilizer by removing the cover layer (258). As shown in FIG. 11B, the sterilizer (270) includes the sterilization material (257) formed on the upper substrate (250), and the cover layer (258) adhered to the upper substrate (250) and for covering the sterilization material (257).

The sterilization material (257) may include at least one or more components selected from a group consisting of sterilizer, antibiotic, biocide, anesthetic, peroxidic sterilizer, halogen sterilizer and alcoholic sterilizer. The peroxidic sterilizer includes hydrogen peroxide, sodium preborate, potassium permanganate, benzoly peroxide and peroxyacetic acid, and 2.5-3.5% of hydrogen peroxide solution is largely used for the peroxidic sterilizer.

The halogen sterilizer may include chlorine or iodine which oxidizes cell membranes of microorganisms and protein of protoplasm to perform the sterilization and disinfection effect against various microorganisms. 2% of iodine solution or 9-12% of povidone iodine solution is largely used for the halogen sterilizer.

The alcoholic sterilizer may include ethanol and isopropanol, and 70% of ethanol is largely used for alcoholic sterilizer. The alcohol for disinfection has a strong osmotic power to easily penetrate membranes of surfaces of bacteria. The ethanol can penetrate the bacteria membranes to coagulate the protein of bacteria or transform the cell membranes of bacteria to kill the bacteria for disinfection.

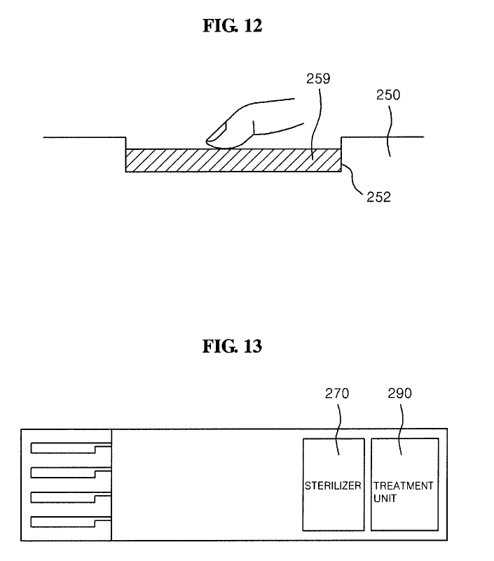

Referring to FIG. 12, the sterilizer formed on the upper substrate of the bio chip is accommodated with a mesh structure (259) for absorbing the sterilization materials. The mesh structure (259) may be soft feeling non-woven fabric, gauze or absorbent sanitary cotton. In other words, if the mesh structure (259) is laid on the sterilizer, the sterilization materials are absorbed by the mesh structure (259), and the sterilization materials are leaked only if there is any outside pressure. Therefore, unless a user's finger touches the mesh structure (259) to allow pressure thereof to be transferred to the mesh structure (259), the sterilization materials are not leaked to prevent the bio chip from being polluted. In so doing, the user can enhance the touch feeling for sterilization just by touching and pressing the mesh structure (259).

Referring to FIG. 13, the bio chip according to the third implementation may include a sterilizer (270) and a treatment unit (290). The user may sterilize the vulnus caused by the blood collection using the sterilizer (270) and cure the vulnus using the treatment unit of the bio chip. The treatment unit (290) is shown to include a groove formed on the upper substrate, and a treatment material filled inside the groove. In one implementation, the treatment material is humectant that provides an environment conducive to treatment of vulnus.

The treatment material may include at least one or more components selected from the group consisting of glycerin, propylene glycol, butylen glycol, polyethylene glycol, sorbitol, trehalose, sodium PCA, hyaluron acid, collagen and betaine.

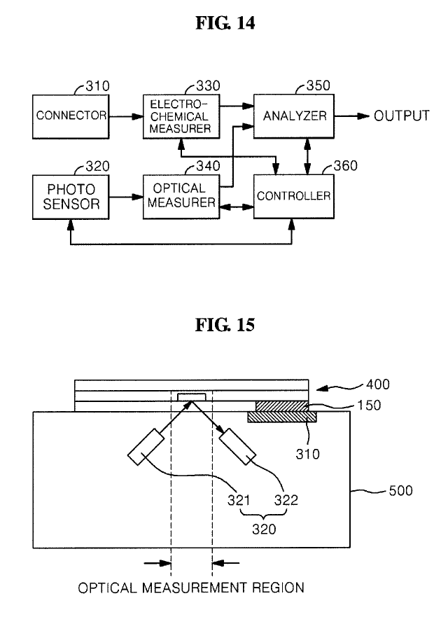

FIG. 14 is a schematic block diagram illustrating an apparatus for analyzing biological material of a bio chip. A connector (310), a photo sensor (320) or both the collector and the photo sensor may be needed in order to assay the biological materials injected into the bio chip according to the first, second and third implementations.

In other words, the connector (310) is brought into contact with the electrode pads formed at the bio chip for electro-chemically measuring the reaction degrees of the specific materials and reaction-inducing materials included in the biological materials.

The photo sensor (320) may irradiate light to through holes in which the specific materials and reaction-inducing materials contained in the biological materials react, and may receive the light that has passed through or that has been reflected from the through holes.

In so doing, a voltage may be applied to the electrode pads of the bio chip via the connector (310). A current variation value in response to the applied voltage may be measured by an electro-chemical measurer (330). The electro-chemical measurer (330) may convert the current variation value to an electrical signal and output the electrical signal.

The photo-sensor (320) may irradiate light to the through holes of the bio chip. Light is received that has passed through or that has been reflected from a region in which the specific materials and reaction-inducing materials contained in the biological materials react. An intensity of the received light may be measured by an optical measurer (340). The light intensity is converted to an electrical signal which is then outputted.

The signals outputted from the electro-chemical measurer (330) and the optical measurer (340) may be inputted into an analyzer (350). The analyzer (350) may perform qualitative and quantitative analyses using the signals inputted from the electro-chemical measurer (330) and the optical measurer (340). The photo sensor (320), the electro-chemical measurer (330), the optical measurer (340) and the analyzer (350) may be controlled by a controller (360).

Therefore, the apparatus for analyzing biological material may comprise: a connector (310) connected to electrode pads of bio chip formed with reaction regions in which specific materials and reaction-inducing materials included in the biological material are reacted, formed with distal ends of electrode lines on part of the reaction regions and having electrode pads connected to the electrode lines; an electro-chemical measurer (330) applying a voltage to the electrode pads of the bio chip via the connector (310) to measure a current variation value in response to the applied voltage, converting the current variation value to an electrical signal and outputting the electrical signal; a photo sensor (320) irradiating light on reaction regions where the distal ends of the electrode lines of the bio chip are not formed, and collecting the light reflected or transmitted therefrom; an optical measurer (340) measuring a light intensity collected from the photo sensor (330), converting the light intensity to an electrical signal and outputting the electrical signal; and an analyzer (350) receiving the signal outputted from the electro-chemical measurer (330) and the optical measurer (340) to qualitatively and quantitatively analyze the biological material. The apparatus may further include a display for displaying an analytical result of the biological materials outputted from the analyzer (350) and storage for storing the analytical result.

The analyzer (350) may include a function capable of analyzing concentration of the specific material contained in the biological materials using the signals outputted from the electro-chemical measurer (330) and the optical measurer (340).

Meanwhile, the apparatus for analyzing biological material may be constructed by mounting the afore-mentioned bio chip to the analyzer of the biological materials.

In other words, the apparatus for analyzing biological material may include: a bio chip formed with reaction regions in which specific materials and reaction-inducing materials included in the biological material are reacted; and a biological material analyzer formed with the bio chip for measuring the reaction regions of the bio chip to qualitatively and quantitatively analyze the biological material, wherein the biological material analyzer comprises: the photo sensor (320) irradiating light on the reaction regions and collecting the light transmitted or reflected therefrom; the optical measurer (340) measuring a light intensity collected from the photo sensor, converting the light intensity to an electrical signal and outputting the electrical signal; and the analyzer (350) receiving the signal outputted from the electro-chemical measurer and the optical measurer to qualitatively and quantitatively analyze the biological material.

The bio chip may further include electrode lines, and electrode pads connected to the electrode lines. A part of the reaction regions may be formed with distal ends of the electrode lines. The biological material analyzer may be further included with a connector (310), and an electro-chemical measurer (330) for measuring a current variation value of a voltage applied to the electrode pads of the bio chip via the connector (310), for converting the current variation value to an electrical signal and outputting the electrical signal.

Furthermore, the analyzer (350) may further receive the signal outputted from the electro-chemical measurer (330) to qualitatively and quantitatively analyze the biological material.

FIG. 15 is a schematic partial cross-sectional view illustrating a state of an apparatus for analyzing biological material of a bio chip, where the apparatus may be formed with a bio chip for analyzing the biological materials or a construction capable of mounting the bio chip.

As shown, the apparatus includes a case (500), where the connector (310) is exposed to the case (500).

In other words, the connector (310) exposed to the case (500) is a mounting unit capable of mounting a bio chip (400) to which the bio chip (400) may be mounted. The electrode pad (150) of the bio chip (400) may be brought into contact with the connector (310) to electro-chemically analyze the biological materials.

The case (500) may be disposed therein with a circuit substrate formed with an electro-chemical measurer and an analyzer, where the electro-chemical measurer may be connected to the connector (310).

In an alternative configuration, the case (500) may be formed with a transparent window. The case (500) may be formed therein with a photo sensor (320) for irradiating light to the transparent window and collecting the irradiated light. Therefore, the photo sensor (320) and the transparent window are able to optically analyze the biological materials. The photo sensor may include a light emitting unit (321) for irradiating light and a light receiving unit (322) for receiving the irradiated light. The case (500) may be provided therein with a circuit substrate formed with an optical measurer and an analyzer. Furthermore, all the components for electro-chemically and optically measuring the biological materials may be provided on a surface of the case or within the case. The light emitting unit (321) may be an LED (Light Emitting Diode) or an LD (Laser Diode) for emitting a single wavelength light.

For example, if the reaction-inducing material that reacts with the biological material is changed to blue, and if a red light is irradiated to the reaction region, the degree of the red light absorbed into the reaction-inducing material may be changed in response to the degree changed to the color of blue. The greater the degree the reaction-inducing material is changed to, the smaller the intensity of light reflected from or passed through the reaction-inducing material. The light receiving unit (322) receives the light reflected from or passed through the reaction-inducing material.

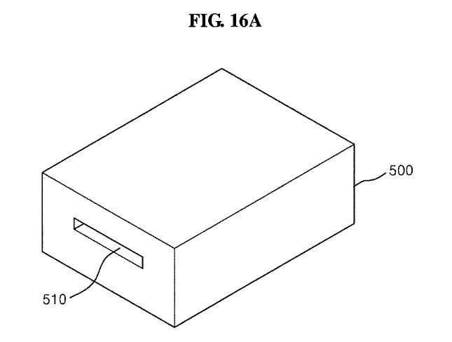

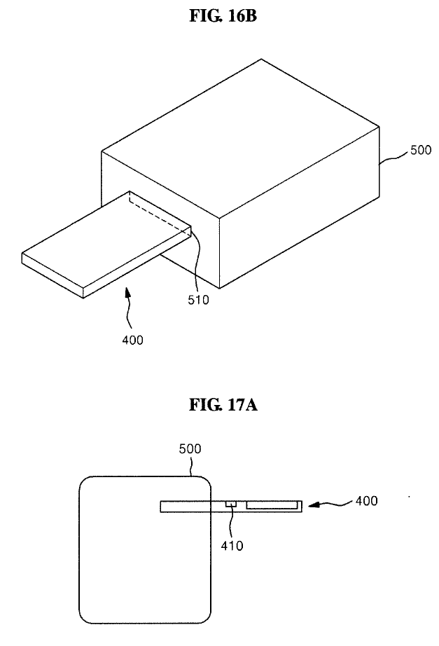

FIGS. 16A and 16B are schematic perspective views illustrating another state of an apparatus for analyzing biological material of a bio chip, in which the case (500) for analyzing the biological materials may be provided with an inserter (510. see FIG. 16A). The inserter (510. see FIG. 16B) may be inserted a part of the bio chip (400) to analyze the biological materials.

The electro-chemical measurer, the optical measurer and the analyzer of FIG. 14 may be formed on a single printed circuit board to be accommodated inside the case. The photo sensor and the connector may be also housed inside the case.

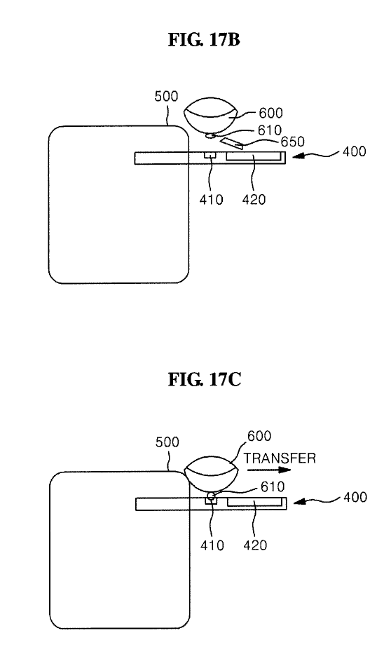

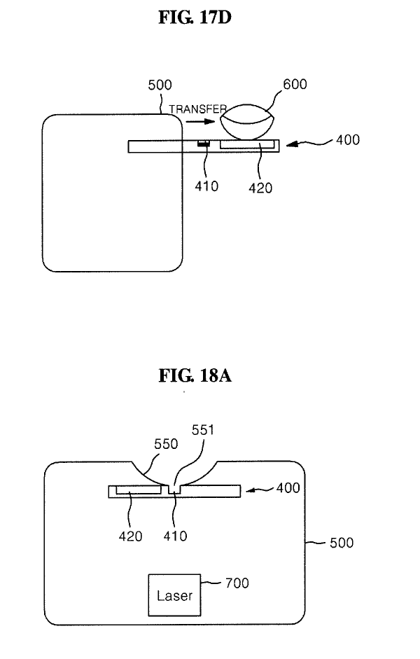

FIGS. 17A to 17D are schematic cross-sectional views illustrating a method for collecting blood from a bio chip of the second implementation immobilized on an apparatus for analyzing biological material.

First of all, as shown in FIG. 17A where the bio chip (400) is mounted on the case (500) for analyzing the biological materials, blood is collected from a finger (600) using a lancet (650) as illustrated in FIG. 17B. Thereafter, blood (610) oozes out from the finger (600) by the blood collection, and the blood (610) is injected into the inlet port (410) of the bio chip (400) as depicted in FIG. 17C. Successively, a user moves the finger (600) to the sterilizer (420) and disinfects the vulnus caused by the blood collection as illustrated in FIG. 17D.

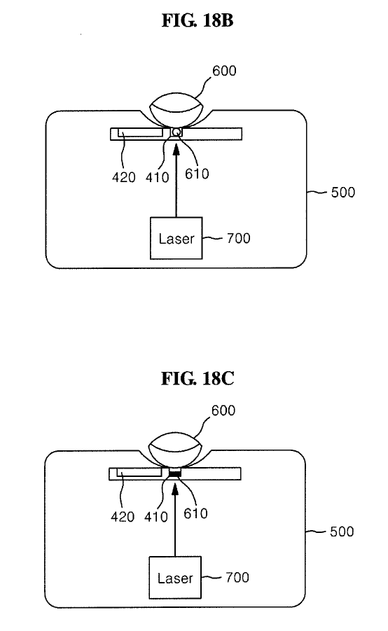

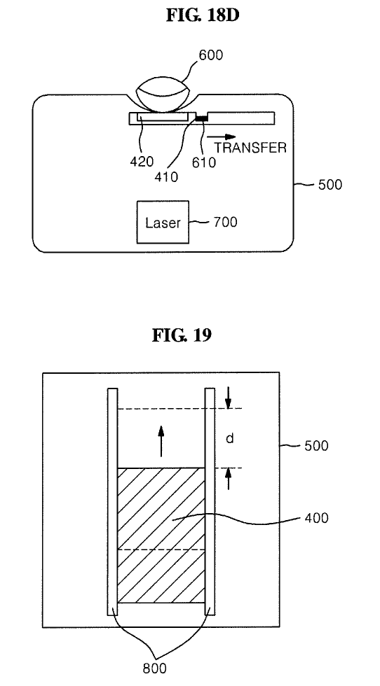

FIGS. 18A to 18D are schematic cross-sectional views illustrating a method for collecting blood from a bio chip of the second implementation immobilized on an apparatus for analyzing another biological material.

A set-up is prepared in such a manner that a concave unit (550) having an opening (551) is formed in the case (500), a transfer unit (not shown) capable of transferring the bio chip (400) is disposed inside the case (500), the bio chip (400) is mounted at the transfer unit, and an apparatus is formed for analyzing the biological materials mounted with a laser beam source (700) for emitting laser beam to the opening (551) of the concave unit (550) formed in the case (500) (see FIG. 18A), where the laser beam source (700) is blood collecting means.

Successively, when a user positions a finger (600) inside the concave unit (550) formed at the case (500), the laser beam source (700) emits laser beam to collect blood (600) through the opening (551) of the concave unit (550) (FIG. 18B). The blood (610) that has oozed out from the finger (600) is injected into the inlet port (410) of the bio chip (400) (FIG. 18C).

Thereafter, the bio chip (400) is transferred to the transfer unit to allow the sterilizer (420) of the bio chip (400) to be positioned at the opening (551) of the concave unit (550), and the vulnus of the finger (600) is disinfected by being brought into contact with the sterilizer (420) (FIG. 18D).

As noted from the above description, there is an advantage in the apparatus for analyzing biological materials in that a laser beam source is mounted at the apparatus to enable a swift blood collection, where the apparatus is provided with a transfer unit for transferring the bio chip having a sterilizer, and where the bio chip is transferred following the blood collection to automatically perform the blood collection, sterilization and analysis.

FIG. 19 is a schematic concept representation illustrating an operation of transferring a bio chip from an apparatus for analyzing biological material, where a transfer rail (800) is formed inside the case (500) of the apparatus for analyzing the biological materials to a transfer unit, and the transfer rail (800) is mounted with the bio chip (400).

Furthermore, as illustrated in FIG. 18D, if the transfer rail (800) is operated to disinfect the finger, the bio chip (400) is moved as long as ‘d’ from a solid line to a dotted line as shown in FIG. 19. As a result, the bio chip is automatically moved to enable a disinfection of the vulnus on the finger caused by the blood collection.

The above-described implementations are not intended to be limited by any of the details of the foregoing description, unless otherwise specified, but rather should be considered broadly to define concepts and specific examples.

What is claimed is:

1. A bio chip for analyzing biological material comprising:
a substrate;
a protection film, positioned on the substrate, structured to define first through holes that expose the substrate, micro channels each connected to the first through holes, and an inlet port structured to receive injected biological materials and connected to the micro channels; and
reaction-inducing materials immobilized on portions of the substrate positioned at and exposed by the first through holes.

2. The bio chip as claimed in claim 1, further comprising:
at least one electrode pad positioned on the substrate;
electrode lines connected to the electrode pad, wherein the protection film is structured to define at least one second through hole that exposes a first portion of the electrode pad and micro channels connecting the second through hole with the inlet port.

3. The bio chip as claimed in claim 2, wherein the second through hole defined by the protection film exposes distal ends of the electrode lines connected to a second portion of the electrode pad.

4. The bio chip as claimed in claim 2, wherein width of each micro channel is in the range of 0.1 mm˜1 mm.

5. The bio chip as claimed in claim 2, wherein the micro channels are positioned inside the protection film or on an upper surface of the protection film.

6. The bio chip as claimed in claim 2, wherein the inlet port is formed at a lateral surface of the protection film.

7. The bio chip as claimed in claim 2, wherein the protection film comprises:
an isolation film positioned on the substrate; and
a polymer film formed on the isolation film,
wherein the isolation film and polymer film are structured and relatively oriented to define the first and second through holes passing there through, and wherein the micro channels are positioned at one side of the polymer film.

8. The bio chip as claimed in claim 2, further comprising an upper substrate positioned on the protection film.

9. The bio chip as claimed in claim 8, further comprising a sterilizer positioned at an upper surface of the upper substrate.

10. The bio chip as claimed in claim 9, wherein the sterilizer comprises:
a groove positioned at the upper surface of the upper substrate; and
sterilization material inside the groove.

11. The bio chip as claimed in claim 10, wherein the sterilizer further comprises a cover layer at least partially covering the groove and adhered to the upper substrate.

12. The bio chip as claimed in claim 9, wherein the sterilizer comprises:
sterilization material positioned at an upper surface of the upper substrate; and
a cover layer at least partially covering the sterilization material and adhered to the upper substrate.

13. The bio chip as claimed in claim 10, further comprising a mesh structure positioned within the groove and capable of absorbing the sterilization material.

14. The bio chip as claimed in claim 10, wherein the sterilization material comprises at least one or more components selected from a group consisting of sterilizer, antibiotic, biocide, anesthetic, peroxidic sterilizer, halogen sterilizer and alcoholic sterilizer.

15. The bio chip as claimed in claim 9, further comprising a treatment unit positioned at the upper surface of the upper substrate.

16. The bio chip as claimed in claim 15, wherein the treatment unit comprises:
a groove positioned on an upper surface of the upper substrate; and
a sterilization material inside the groove.

17. The bio chip as claimed in claim 16, wherein the treatment material comprises at least one or more components selected from a group consisting of glycerin, propylene glycol, butylen glycol, polyethylene glycol, sorbitol, trehalose, sodium PCA, hyaluron acid, collagen and betaine.

18. A bio chip for analyzing biological material comprising:
a substrate;
at least one electrode pad positioned at an upper surface of the substrate;
electrode lines connected to the electrode pad;
a protection film, positioned at an upper surface of the substrate, and structured to define through holes that expose the electrode pad, and to define micro channels each connecting the through holes to an inlet port into which biological materials are injected; and
reaction-inducing materials immobilized on the distal ends of the electrode lines exposed to each through hole.

19. An apparatus for analyzing biological material comprising:
a connector connected to electrode pads of bio chip including:
reaction regions in which reaction-inducing materials and introduced biological material are reacted,
electrode lines with distal ends extending to the reaction regions, and
electrode pads connected to the electrode lines;
an electro-chemical measurer configured to apply a voltage to the electrode pads of the bio chip via the connector, to measure a current variation value in response to the applied voltage, to convert the current variation value to an electrical signal, and to output the electrical signal;
a photo sensor configured to irradiate light on reaction regions at locations where the distal ends of the electrode lines are absent, and collecting light reflected or transmitted therefrom;
an optical measurer configured to measure a light intensity collected from the photo sensor, convert the light intensity to an electrical signal and output the electrical signal; and
an analyzer configured to receive the signal outputted from the electro-chemical measurer and the optical measurer and to qualitatively and quantitatively analyze the biological material.

20. An apparatus for analyzing biological material comprising:
a bio chip including reaction regions in which specific materials and reaction-inducing materials within the biological material are reacted;
a biological material analyzer configured to measure aspects of reactions occurring at the reaction regions of the bio chip to qualitatively and quantitatively analyze the biological material, wherein the biological material analyzer comprises: a photo sensor irradiating light on the reaction regions and collecting the light transmitted or reflected therefrom;
an optical measurer configured to measure a light intensity collected from the photo sensor, to convert the light intensity to an electrical signal and to output the electrical signal; and
an analyzer configured to receive the signal outputted from the electro-chemical measurer and the optical measurer and to qualitatively and quantitatively analyze the biological material.

21. The apparatus as claimed in claim 20, wherein the bio chip further includes electrode lines and electrode pads connected to the electrode lines, and a part of the reaction regions includes distal ends of the electrode lines, and wherein the biological material analyzer further comprises:
a connector; and
an electro-chemical measurer configured to measure a current variation value of a voltage applied to the electrode pads of the bio chip via the connector and for converting the current variation value to an electrical signal and outputting the electrical signal, and wherein the analyzer further receives the signal outputted from the electro-chemical measurer to qualitatively and quantitatively analyze the biological material.

22. The apparatus as claimed in claim 21, wherein the electro-chemical measurer, the optical measurer, the analyzer and the photo sensor are housed within a single case, and the connector is housed within the case or on a surface of the case.

23. The apparatus as claimed in claim 21, wherein the electro-chemical measurer, the optical measurer, the analyzer, the photo sensor and the connector are housed within a single case, and wherein the case is provided with an inserter into which a part of the bio chip is inserted.

24. The apparatus as claimed in claim 23, wherein the bio chip is formed with one of the sterilizer and the treatment unit or both the sterilizer and the treatment unit.

25. The apparatus as claimed in claim 21, wherein the electro-chemical measurer, the optical measurer, the analyzer, the photo sensor and the connector are housed within a single case, a concave unit having an opening located in the case, and a laser beam source for emitting a laser beam to the opening of the concave unit formed in the case.

26. The apparatus as claimed in claim 25, wherein a transfer unit is formed inside the case, and the bio chip is mounted at the transfer unit.
